(19) United States
(12) Patent Application Publication
(10) Pub. No.: US 2016/0287640 A1
(43) Pub. Date: Oct. 6, 2016

(54) DENUDED AMNION FLOWABLE TISSUE GRAFT AND METHOD OF FORMING SAME

(71) Applicant: Applied Biologics, LLC, Scottsdale, AZ (US)

(72) Inventor: Edward Britt, Scottsdale, AZ (US)

(21) Appl. No.: 14/986,436

(22) Filed: Dec. 31, 2015

Related U.S. Application Data
(60) Provisional application No. 62/098,994, filed on Dec. 31, 2014.

(51) Int. Cl. A61K 35/50; A61K 35/28

(52) U.S. Cl. CPC A61K 35/50; A61K 35/28

(57) ABSTRACT

The invention relates to preparations and methods of creating preparations of reconstituted amniotic membrane denuded of the amniocyte/epithelial cell layer to preserve amniocytes viability during processing. The tissue graft utilizes amniotic fluid as a source of additional viable stem cells. The amniotic tissue graft is flowable and injectable, and designed for use in surgical and minimally invasive medical therapy of injury and disease to promote tissue regeneration and healing. The tissue grafts optimize available quantities of viable mesenchymal stem cells and non-cellular bioactive compounds to enhance therapeutic efficacy. The tissue grafts are semi-viscous fluids which may be intraoperatively transplanted at the recipient site using a needless syringe or by non-operative percutaneous injection through a hypodermic needle.

### CROSS REFERENCE TO RELATED APPLICATION

This application claims priority from U.S. Provisional Application No. 62/098,994, filed Dec. 31, 2014, the contents of which are incorporated entirely herein by reference.

### BACKGROUND OF THE INVENTION

1. Technical Field

This invention relates to the preparation of a denuded amnion flowable tissue graft from an amnion; in particular, the invention relates to a preparation and method of formation of an amniotic tissue graft product comprising variable concentrations of viable embryonic mesenchymal stem cells.

2. State of the Art

Amniotic membrane, specifically human amniotic membrane, has been used in surgery for over one hundred years. The amnion interstitial matrix contains a complex biologic soup of growth factors, inflammatory mediators, immuno-modulators, and other active biomolecules. Additionally, amniotic membrane is rich in embryonic mesenchymal stem cells.

Amniotic membrane is used in a variety of surgical procedures as an adjunct to healing, and to minimize formation of scar tissue and adhesions. The amniotic membrane is often dried prior to packaging, sterilization, and storage. Some preparation, reconstitute the dried amniotic membrane using a tissue preservative solution prior to the packaging and sterilization for storage. The medium used to reconstitute the dried amniotic membrane is typically an isotonic solution containing water and electrolytes, but no growth factors, other active biomolecules, or additional extraembryonic stem cells.

Depending on the preparation methods employed when processing AM for use as a tissue graft, none, some, or all of the amniocytes, which comprise embryonic stem cells, may be viable. The concentration of viable SCs is not standardized in currently available preparations, and is often not known.

Accordingly, what is needed is method of reconstituting a dried amniotic membrane tissue graft which supplants the tissue proliferative, antimicrobial, immuno-modulatory, and anti-inflammatory properties of amniotic membrane and amniotic fluid comprising a known concentration of viable stem cells.

Citation of documents herein is not an admission by the applicant that any is pertinent prior art. Stated dates or representation of the contents of any document is based on the information available to the applicant and does not constitute any admission of the correctness of the dates or contents of any document.

### DISCLOSURE OF EMBODIMENTS OF THE INVENTION

Disclosed is a denuded amnion flowable tissue graft comprising an amniotic membrane stromal matrix denuded of amniocytes; and a non-amniotic fluid liquid, wherein the non-amniotic fluid liquid hydrates the amniotic membrane stromal matrix.

In some embodiments, the tissue graft further comprises an amniotic fluid derivative. In some embodiments, the tissue graft further comprises a non-denuded amniotic membrane having a stromal matrix and an amniocyte layer. In some embodiments, the denuded amniotic membrane is sonicated. In some embodiments, the non-denuded amniotic membrane is morcellized. In some embodiments, the non-denuded amniotic membrane is ground. In some embodiments, the non-amniotic fluid liquid is an isotonic electrolyte solution, a cryoprotectant, or both an isotonic electrolyte solution and a cryoprotectant.

Disclosed is a denuded amniotic membrane flowable tissue graft comprising an amniotic membrane stromal matrix denuded of amniocytes; an amniotic fluid derivative, wherein the amniotic fluid derivative hydrates the amniotic membrane stromal matrix; and a sonicated amnion suspension.

In some embodiments, the denuded amnion flowable tissue graft, the sonicated amnion suspension, the amniotic fluid derivative, or both the sonicated amnion suspension and the amniotic fluid derivative comprises a known concentration of viable mesenchymal stem cells. In some embodiments, the concentration of viable mesenchymal stem cells is less than 5.0×10⁵/ml In some embodiments, the concentration of viable mesenchymal stem cells is between 5.0×10⁵/ml and 1.0×10⁶/ml. In some embodiments, the concentration of viable mesenchymal stem cells is between 5.0×10⁵/ml and 1.50×10⁶/ml. In some embodiments, the concentration of viable mesenchymal stem cells is between 1.0×10⁶/ml and 1.50×10⁶/ml. In some embodiments, the concentration of viable mesenchymal stem cells is between 5.0×10⁵/ml and 7.5×10⁵/ml. In some embodiments, the concentration of viable mesenchymal stem cells is between 7.5×10⁵/ml and 1.0×10⁶/ml. In some embodiments, the concentration of viable mesenchymal stem cells is between 1.0×10⁶/ml and 1.250×10⁶/ml. In some embodiments, the concentration of viable mesenchymal stem cells is between 1.25×10⁶/ml and 1.5×10⁶/ml. In some embodiments, the concentration of viable mesenchymal stem cells is between 7.4×10⁵/ml and 7.6×10⁵/ml. In some embodiments, the concentration of viable mesenchymal stem cells is greater than 1.5×10⁶/ml.

Also disclosed is a denuded amnion flowable tissue graft comprising an amniotic membrane stromal matrix denuded of amniocytes; a non-denuded amniotic membrane comprising an amniocyte layer; and a non-amniotic fluid liquid, wherein the non-amniotic fluid liquid hydrates the amniotic membrane stromal matrix. In some embodiments, the denuded amniotic membrane is sonicated. In some embodiments, the non-denuded amniotic membrane is morcellized. In some embodiments, the non-denuded amniotic membrane is ground.

In some embodiments, the invention includes a set of tissue grafts, wherein each tissue graft in the set comprises a denuded amnion flowable tissue graft that includes an amniotic membrane stromal matrix denuded of amniocytes, an amniotic fluid derivative that hydrates the amniotic membrane stromal matrix, and a sonicated amniotic suspension. In such embodiments of the invention, either the sonicated amniotic suspension, the amniotic fluid derivative, or both the sonicated amniotic suspension and the amniotic fluid derivative comprise a known quantity of viable mesenchymal stem cells such that the tissue graft contains a known concentration of viable mesenchymal stem cells. For example, in various embodiments, the concentration of viable mesenchymal stem cells in each denuded amnion flowable tissue graft in the set of tissue grafts is less than 5.0×10⁵/ml, between 5.0×10⁵ and 1.50×10⁶/ml, between 5.0×10⁵ and 7.5×10⁵/ml, between 7.5×10⁵ and 1.0×10⁶/ml, between 1.0×10⁶ and 1.250×10⁶/ml, between 1.25×10⁶ and 1.5×10⁶/ml, between 7.4×10⁵ and 7.6×10⁵/ml, or greater than 1.5×10⁶/ml.

In some embodiments, the invention includes a set of tissue grafts, where each tissue graft in the set comprises an amniotic membrane stromal matrix denuded of amniocytes, an amniotic fluid derivative that hydrates the amniotic membrane stromal matrix, a sonicated amnion suspension, and a known concentration of viable mesenchymal stem cells. In various embodiments, the concentration of viable mesenchymal stem cells in each tissue graft in the set of tissue grafts is less than 5.0×10⁵/ml, between 5.0×10⁵/ml and 1.50×10⁶/ml, between 5.0×10⁵/ml and 1.0×10⁶/ml, between 1.0×10⁶/ml and 1.50×10⁶/ml, between 5.0×10⁵/ml and 7.5×10⁵/ml, between 7.5×10⁵/ml and 1.0×10⁶/ml, between 1.0×10⁶/ml and 1.250×10⁶/ml, between 1.25×10⁶/ml and 1.5×10⁶/ml, between 7.4×10⁵/ml and 7.6×10⁵/ml, or greater than 1.5×10⁶/ml.

In various embodiments, the invention may comprise a set of tissue grafts that includes a minimum number of tissue grafts. For instance, a set of tissue grafts of the invention may include at least 1, at least 5, at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, or at least 1000 tissue grafts.

Disclosed is a method of forming a denuded amnion flowable tissue graft comprising the steps of morcellizing a first amnion; sonicating a second amnion; preparing an amniotic fluid derivative; and mixing the morcellized first amnion with the sonicated second amnion and the amniotic fluid derivative to form a denuded amnion flowable tissue graft.

In some embodiments, the method further comprises a step diluting the denuded amnion flowable tissue graft with a suitable fluid to form a standardized denuded amnion flowable tissue graft. In some embodiments, the method further comprises a step digesting the second amnion with an enzyme. In some embodiments, the enzyme is trypsin.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a representation of a denuded amnion flowable tissue graft 100;

FIG. 2 is a schematic diagram showing overview 200 of processing steps used in forming some embodiments of denuded amnion flowable tissue graft 100;

FIG. 3 is a schematic diagram of processing step groups utilized in forming some embodiments of denuded amnion flowable tissue graft 100; and

FIG. 4 is a schematic diagram showing steps of a method 400 of forming denuded amnion flowable tissue graft 100.

### DETAILED DESCRIPTION OF EMBODIMENTS OF THE INVENTION

Fetal placental membranes (“PMs”) occupy a unique position in the field of regenerative medicine. This tissue, which derives solely from the developing embryo and fetus, comprises amnion (amniotic membrane or “AM”) and chorion (chorionic membrane or “CM”) fused at a basement-membrane/stroma interface and contains a dense concentration of extraembryonic mesenchymal stem cells (“SCs”) in an interstitial matrix rich with multiple classes of biologically active molecules.

The AM is a single layer of epithelial cells—amniocytes—on a thick basement membrane/connective tissue stroma. It derives from the embryonic epiblast, which is adjacent to the primitive streak and contiguous with cells giving rise to the notochord, and grows into a fluid-filled sac enveloping the developing fetus.

The CM is a more complex tissue, adjacent to and invading the maternal uterine wall, but arising from the embryonic trophoblast. In contrast to the histologically simple amniotic membrane, the chorion is more complex. The trophoblast is a tissue on the uterine surface of the chorion and contains subpopulations of cells. One cell population, the extravillous cytotrophoblast, invades the maternal endometrium. Another, the syncytiotrophoblast, forms a syncytium of densely nucleated cytoplasm covering the chorionic villi and directly contacting the maternal blood. Like the AM, the CM is also rich in undifferentiated extraembryonic mesenchymal stem cells. Unlike the AM, CM is used less extensively as a tissue graft because of its immunogenicity. This arises from residual bits of decidua (maternal endometrial tissue contacting the placenta). Additionally, and perhaps more importantly, CM tissue components of fetal origin, including fetal blood vessels, connective tissue, endothelial cells, and residual fetal blood elements, elicit an immunological response in the tissue graft recipient leading to rejection of the tissue graft. And although the CM stromal layer, which is adjacent to the basement membrane of the AM, contains non-immunogenic SC's and large/small biomolecules, the trophoblast and fetal connective tissue components express HLA Class I and HLA-D cell surface antigens which allow development of a full host immune response to grafted CM. Consequently, intact AM which is manually “peeled” from the AM at the stromal interface is used in various tissue graft preparations whereas use of CM is limited by its antigenicity. The CM is a source of beneficial tissue, SCs, and biomolecules. When the placental membranes are received from a volunteer donor and the CM is discarded, however, at least half of the donor's PM SCs and bioactive molecules are lost.

Amniotic fluid (“AF”) is an additional source of beneficial material. AF, which bathes the fetus and is contained by the AM, is a biologically complex substance which, although extensively studied, remains incompletely defined and understood. It is known that AF contains large numbers of suspended amniocytes, SCs, and non-cellular components including small molecules, growth factors, hormones, immunomodulators, and antimicrobials. Small molecules in solution within the AF include electrolytes, glutamine (important for nucleic acid synthesis), arginine (necessary for placental angiogenesis), and hyaluronic acid (inhibits collagen synthesis; may mitigate scaring and fibrosis during wound healing). Growth factors identified in AF include transforming growth factor alpha (“TGF-α”), epidermal growth factor (“EGF”), insulin-like growth factor I (“IGF-1”), hyaluronic acid-stimulating factor, macrophage colony-stimulating factor (“M-CSF”), and granulocyte colony-stimulating factor (“G-CSF”). These growth factors all potently stimulate proliferation of stem cell and many non-progenitor cell-types in both fetal and adult cells and tissues. Hormones identified in AF include erythropoietin (“EPO”), which promotes proliferation of red blood cell progenitors and may stimulate growth of the gut endothelium. Immunomodulators and antimicrobials in AF include α-defensins, lactoferrin, lysozyme, bactericidal/permeability-increasing protein, calprotectin, secretory leukocyte protease inhibitor, psoriasin, a cathelizidin, and various polyamines with antimicrobial properties. Additionally, cellular immune components present in AF include monocytes, macrophages, and histiocytes. In addition to all of these substances, AF almost certainly contains additional compounds which also provide benefits to a graft recipient.

Although AF contains many phenotypically distinct subpopulations of SCs, these cells generally do not express HLA Class I, II, and other cell surface antigens in a manner sufficient to elicit a host immune response, as measure by a mixed lymphocyte reaction (“MLR”). Therefore, AF is a valuable source of biologically active molecules and immune-privileged pluripotent SCs.

Collection of AF for preparation of tissue grafts during the peri-partum period in the time prior to a vaginal delivery is not possible. Amniocentesis under sterile, controlled conditions prior to the peri-partum period is source of sterile AF. Amniocentesis, however, when performed to obtain a tissue donation should not justify even a small risk to the developing fetus. Amniocentesis carries a risk of spontaneous abortion of up to 0.5% when electively performed in the second trimester. Consequently, AF during pregnancy cannot, safely and practically, be collected in significant bulk from a pool of volunteer donors prior to or during a vaginal delivery.

Amniotic fluid may be collected under sterile conditions in the operating room during an elective Cesarean section delivery with essentially no risk to the infant or the mother. There are just under 4 million births per year in the United States of which approximately 33%—1.32 million overall—are by Cesarean delivery. Fetal placental membranes, including AM and CM, however, may additionally be collected during a routine vaginal delivery. The bacterial contamination that occurs with vaginal delivery of the placenta is minimal in an uncomplicated delivery and may be addressed. Fetal membranes use as tissue grafts collected from a vaginally delivered placenta may be effectively treated with sterile washings using topical antibiotic and non-tissue-toxic antimicrobial solutions immediately following delivery and thereafter. Therefore, AM but not AF is potentially available for use as an injectable amniotic tissue graft from between 3.5 and 4.0 million births annually in the U.S.

Conversely, AM suitable for use in an injectable amniotic tissue graft is not universally available through a Cesarean delivery where suitable AF is obtained. Gross contamination rendering the AM unsuitable for grafting may occur during the delivery itself, or later during processing and/or packaging.

As briefly mentioned, AM may be collected from suitable volunteer donors and processed for storage prior to use as a tissue injectable amniotic tissue graft in a variety of surgical procedures. AM is used in a plethora of surgical procedures and non-surgical applications. Some examples include use of AM as a biologic dressing, an adjunct to healing of surgically repaired bone, tendon, other soft tissue, and open wounds; a means to militate the formation of scar tissue and adhesions, and other beneficial applications in surgery and non-surgical minimally invasive medical therapies. AM and AM derivatives are used as biologic dressings containing a source of SCs and growth factors to treat burns, skin pressure ulcers, other chronic open wounds, corneal ulcers, and as a dressing following corneal transplant and other ocular procedures. AM tissue injectable amniotic tissue grafts are used to address soft tissue defects and facilitate healing following debridement and repair of damaged cartilage, tendon, bone, and muscle tissue. AM is under investigation as a connective tissue scaffolding for tissue and organogenesis using extraembryonic SCs and other progenitor cells.

In all of these and other applications, there is strong evidence that the presence of viable SCs and active biomolecules in the AM-derived dressing or tissue graft improves healing across a broad range of tissue types, locations within the body, and applications. Reporting of clinical results may eventually lead to the use of AM and AM-derived preparations as a standard therapy and possibly even a best practice for the treatment of a variety of conditions. Such reporting requires continued laboratory experimentation and human clinical trials to generate additional data for review and interpretation in light of currently available practices and results therefrom. Meaningful interpretation of these data however, depends on reproducibility. Reproducibility requires standardization of materials and techniques. Such standardization in this area should include the delivered dose of SCs, total tissue weight per volume, and the concentration of small and large-molecule biologically active compounds present in the tissue graft used.

Preparation and sterilization of AM for later use as an injectable amniotic graft typically includes drying, packaging, sterilization, and storage. Drying discourages bacterial growth and helps maintain sterility during storage. Drying, however, has negative effects on AM. Drying may be accomplished by heating or freezing in a partial vacuum (lyophilization or “freeze drying”) to minimize water-ice crystal formation and cellular disruption. Although some viable SCs are preserved by drying under controlled conditions (use or a suitable cryoprotectant combined with controlled-rate freezing) other SC's die during processing. It is not fully known how drying and storage affect the concentration of the biologically active non-cellular components of AM, although decreasing rates of SC viability are described with progressively longer storage times. Additionally, a significant decrease in concentration of intact proteins and other large biomolecules is possible with prolonged storage. Sterilization by heat or radiation destroys the cellular components of AM preparations, including SCs. Thermal or irradiative sterilization methods may also denature proteins and alter or destroy other large biologically active molecules.

One way of preserving SC viability is by separating the SCs (amniocytes) from the AM prior to tissue milling, grinding, morcellizing, or other mechanical processing of the AM. Mechanical, enzymatic, or a combination of mechanical and enzymatic means may be employed to separate SCs from the stromal component of the AM.

Some tissue graft preparations reconstitute the dried AM using a tissue preservative solution prior to packaging and storage. The medium used to reconstitute the dried amniotic membrane is typically a buffered isotonic solution containing water and electrolytes, but no growth factors, other active biomolecules, or additional SCs. And although the dimensions and weight of dried AM may be easily measured and recorded in the available graft tissue, the absolute number and concentration of viable SCs per unit weight or volume of tissue, which may prove to have high clinical relevance for optimal dosing, is not known by the patient-treating provider.

It is beneficial to know the number of viable stem cells per unit dose (by weight or volume) of injectable amniotic tissue graft. For some uses, a tissue graft preparation containing growth factors, inflammatory modulators, and other biologically active molecules without supplemental SCs is adequate. For such therapeutic applications wherein viable stem cells add little to the efficacy of the treatment, preparations containing a low concentration of viable SCs per ml can be used, reserving higher numbers of viable stem cells for other uses. Examples of such applications are include the non-invasive or minimally-invasive treatment of entero-cutaneous, entero-vaginal, entero-enteric, broncho-pleural, tracheal-esophageal fistulas; treatment of wound sinus tracts; treatment of micro-fractures and small facial fractures; other facial trauma; chronic inflammatory bursitis; intervertebral facet-based pain; injection into peri-rotator cuff soft tissues following rotator cuff repair; injection to facilitate non-surgical repair and healing of supraspinatus, infraspinatus, teres minor, and subscapularis tears; other muscle, ligament, tendon, and soft-tissue tears; application to entero-entero and other surgical anastomoses; treatment of epicondylitis and other similarly debilitating chronic fascial inflammatory conditions such as plantar fasciitis or fasciolosis; and intra-peritoneal application following surgical adhesiolysis.

For other applications, an injectable amniotic tissue graft comprising a higher concentration of viable SCs may be more useful. In these applications, the presence of medium-to-high concentrations of viable SCs allows for SC engraftment into a host tissue and possible differentiation into host tissue cells, such as chondrocytes, osteocytes, fibroblasts, keratinocytes, and other tissue cells. Some examples of such applications are graft-repair of osteochondral defects in the knee, hop, ankle, wrist, hand, and other joints; filling of large bone tissue void following surgical treatment of certain cancers; grafting of cutaneous and soft-tissue defects resulting from deep thermal or radiation burns; spinal and other bony fusion procedures (when combined with currently available bone putty or as a stand-alone application into a cervical or lumbar intervertebral spacer); bone grafting; alveolar cleft (“cleft palate”) grafting; treatment of dental/tooth tissue defects; tears of the meniscal cartilage; intra-peritenon implantation following Achilles' tendon debridement and anastamotic repair; defects of the calvarium following trauma; emergency decompressive craniotomy; application to joint articular surfaces following acetabular and other articular joint surface resurfacing; and treatment of chronic wounds, radiation burns, and thermal injury by direct application or local injection.

Substantial differences in both the absolute amount and concentration per unit volume of biologically active substances in the final preparation arise in currently available preparations based upon the preparation methods used. Existing AM tissue graft preparations are typically formed by suspending a single deconstructed amnion, whether micronized, morcellized, or shredded, in a suitable fluid. Depending on the processing method used, all, some, or no SCs will remain viable after AM processing. The weight of an individual amnion is also variable. Although recording placental weights may not directly reflect amnion weights, a published study (Lurie, et al., (1999) “Human fetal-placental weight ratio in normal singleton near-term pregnancies” Gynecologic and Obstetric Investigation, 48(3): 155-57) of 431 uncomplicated singleton deliveries revealed a mean placental weight of 613+/−123.8 mg, ranging from 319 mg to 1,266 mg. Thus, the weight of the human placenta and its constituent components commonly ranges by nearly 40% around the mean weight and may vary by as much as 400%. The commonly used techniques in preparation of AM-derived suspensions, therefore, result in a preparation with a completely arbitrary total amount and concentration of AM. In some preparation methods, AM is subject to different degrees of drying, whether intentionally or unintentionally. Random samples of AM processed and stored in non-standardized conditions with respect to temperature and drying time revealed an average weight of 1.02+/−0.12 mg/cm².

What is lacking in the prior art, therefore, is an AM-derived tissue graft preparation incorporating a known, effective concentration of SCs and active biomolecules while minimizing loss during processing and storage of SCs and non-cellular tissue elements available from an individual donor or largest possible pool of volunteer donors in a standardized, reproducible concentration.

Embodiments of this invention address these fundamental AM tissue graft requirements—high concentration of viable SCs and beneficial biomolecules in a standardized preparation with no antigenic material and minimal waste of available donor tissue—by forming a combined tissue graft comprising a preparation of dried particulate AM rehydrated by a reconstituted AF-derived suspension and frozen for storage and transport in standardized concentrations. Viable SCs are preserved by separating amniocytes from the AM stromal layer early in processing, and then later re-introduced into the preparation.

Disclosed is a denuded amnion flowable tissue graft with standardized stem cell component, including a method of forming same, comprising dried and morcellized amniotic membrane and amniocyte suspension reconstituted with an amniotic fluid derivative. In some embodiments, the preparation further comprises a standardized quantity of viable SCs per unit volume, a standardized weight of ground AM per unit volume, or both. The denuded amnion flowable tissue graft preparation is used by medical providers as a standardized flowable amniotic tissue graft, either by intraoperative application or injection, non-operative percutaneous injection, or direct application to injured, ischemic, infected, or otherwise damaged tissue. The preparation is also used by laboratory researchers as a reproducible source of standardized material for basic science research of the effects of injectable amniotic AM/AF preparations on healthy, diseased, and damaged tissue; in the field of regenerative medicine; and in other scientific disciplines. The use of morcellized, dried AM supplanted with a suspension of viable amniocytes and reconstituted with AF, with or without additional isotonically balanced electrolyte solutions and/or cryoprotectant, maximizes delivery of SCs and beneficial biologic substances within a non-antigenic flowable amniotic tissue graft to the treatment site. Further, the use of a tissue graft in some embodiments with a known, standardized concentration of SCs per unit weight of volume facilitates predictability and reproducibility of results in both clinical and laboratory applications and allows for focused use of viable extraembryonic mesenchymal SCs in applications where engraftment of viable progenitor cells improves outcomes.

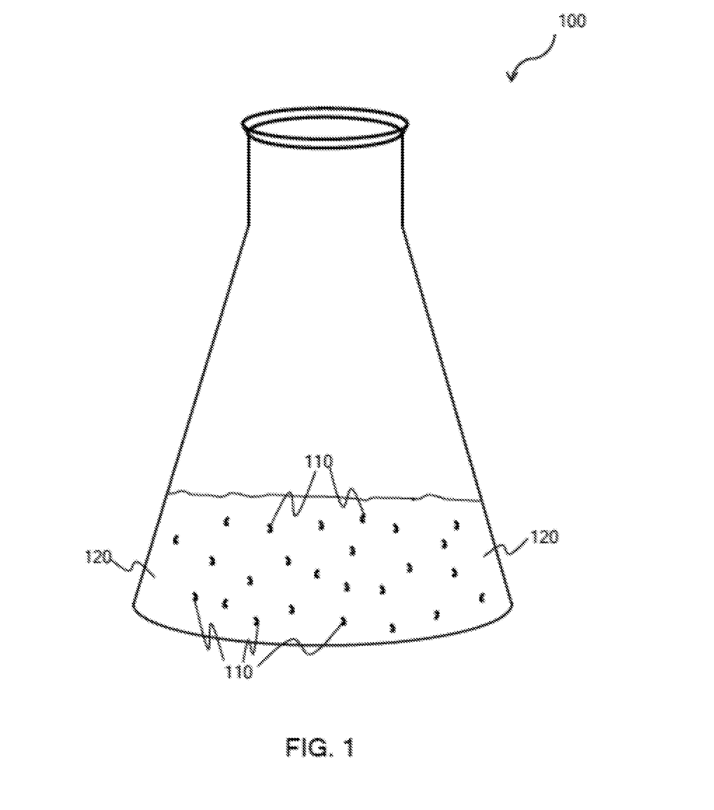

FIG. 1 shows a representation of a denuded amnion flowable tissue graft 100. Details regarding the composition and preparation of denuded amnion flowable tissue graft 100 are provided herein below and throughout this disclosure. Denuded amnion flowable tissue graft 100 comprises fragments of a dried amniotic membrane 110 re-combined with preserved amniocytes and reconstituted with a processed amniotic fluid derivative 120. As is discussed herein below, amniotic fluid derivative 120 rehydrates the dried or partially dried fragments of amniotic membrane 110. In some embodiments, amniotic fluid derivative 120 is fresh amniotic fluid without any processing or addition of other material. In some embodiments, amniotic fluid derivative 120 is a processed amniotic fluid derivative which has been reconstituted with a suitable fluid following serial washings discussed in detail herein below. In some embodiments, amniotic fluid derivative further comprises a sonicated amnion solution (“SAS”) comprising a suspension of viable SCs. In some embodiments, the SAS has a known concentration of viable SCs/ml.

FIG. 1 shows denuded amnion flowable tissue graft 100 contained in a flask. This is only for illustration purposes. Denuded amnion flowable tissue graft 100, in some embodiments, is contained in a variety of packaging means, including sealed single-dose vials, hypodermic syringes, multi-dosed vials, and as a frozen material which is thawed and reconstituted by the addition of a suitable fluid when ready for use, such as a buffered isotonic electrolyte solution for example.

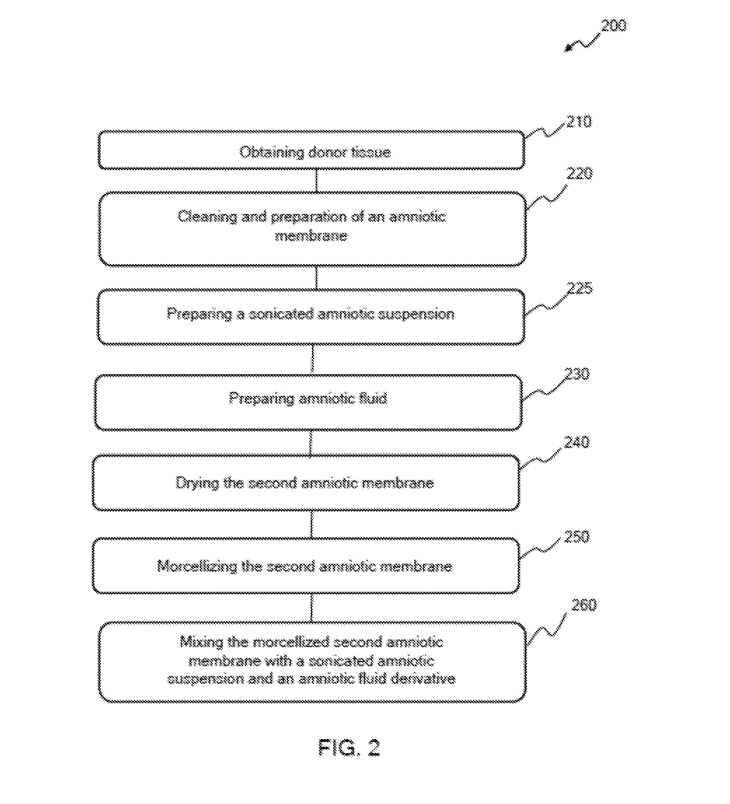

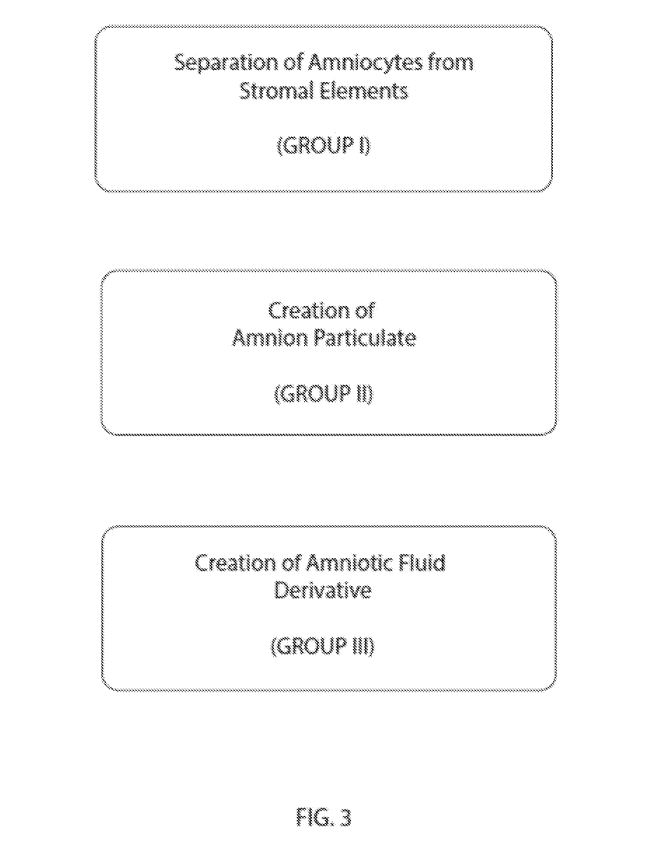

FIG. 2 shows an overview 200 of the processing steps utilized in some embodiments to create a denuded amniotic membrane flowable tissue graft 100. FIG. 3 shows a descriptive grouping of some steps comprising overview 200, discussed further herein below. Overview 200 requires an amnion. In some embodiments of the invention, the AM comes from a volunteer human donor. Accepting amniotic tissue from volunteer donors and excluding non-volunteer and/or paid donors from the donor pool is consistent with internationally well-established tissue donation protocols because it reduces the chance of transmission of infectious agents from the graft donor to the graft recipient. Screening of potential volunteer donors, therefore, includes obtaining a comprehensive past medical and social history, complete blood count, liver and metabolic profile, and serologic testing for HBV, HCV, and HIV, in some embodiments. In some embodiments, other infectious agents are screened for.

In some embodiments, donor tissue is obtained during delivery by elective Cesarean section. In some example embodiments, intraoperative aspiration of AF is performed immediately prior to delivery and the aspirated AF is sealed in a plastic specimen container. Following Cesarean delivery of the infant, the placenta is delivered. The combined fetal membranes (AM and CM) are dissected from the maternal placental plate (decidua). The combined fetal membranes are then placed in a second sterile specimen container and a quantity of 0.9% sterile saline is added sufficient to completely submerge the combined fetal membranes. The individual sterile containers containing the feta placental membranes and amniotic fluid collected under sterile conditions in the operating room are then placed together in a donor tissue specimen bag. This bag is placed within a second sterile bag, sealed, and taken from the operating room for packaging in an insulated ice-bath container. The container is then immediately transported to the processing facility by staff who rotate on call, such that there is minimal delay following delivery before the donor tissue arrives at the separate facility for processing.

Despite the preference for a Cesarean-delivered AM in order to increase the pool of potential donors and other of the aforementioned reasons, vaginally delivered fetal membranes are utilized in some embodiments. Great care must be afforded the vaginally-delivered placental tissue to prevent microbial contamination. Vaginally-delivered fetal membranes are not acceptable donor tissue if there is fecal or other grossly visible contamination, or if there is contact of the placental membranes with clothing, bedding, non-sterile unprepped skin, or other non-sterile surfaces during delivery or prior to sterile packaging. Neither a vaginally-delivered AM nor a Cesarean-delivered AM is acceptable donor tissue if there is visible staining of the fetal membranes with meconium. Following delivery, the steps for preparing vaginally delivered fetal membranes are the same as the above description of preparing Cesarean-delivered fetal membranes. A fully gowned-and-gloved staff member processes the fetal membranes on a sterile field established on a back table, or similar surface, in the labor/delivery room. An additional step comprising rinsing the vaginally delivered fetal membranes with an antimicrobial solution is used in some embodiments. After washing with 0.9% sterile saline, the vaginally delivered dissected fetal membranes are washed with a topical antimicrobial solution. Examples of the topical antimicrobial solution used to wash the vaginally delivered fetal membranes, in some embodiments, are a 0.5% aqueous solution of glutaraldehyde (which is then washed off the donor tissue using a final rinse of 0.9% sterile saline prior to packaging), a Penicillin-Streptomycin solution comprising 50-100 International Units (“IU”) per ml of penicillin and 50-100 micrograms/ml of Streptomycin, or a 0.0125% aqueous solution of sodium hypochlorite. These examples are not meant to be limiting. Other antimicrobial solutions toxic to infectious microorganisms at non-cytotoxic concentrations may also be used. The fetal membranes, following the antimicrobial washing, are then placed in a sterile specimen container, covered with 0.9% sterile saline solution, and sealed in sequential sterile bags as described above for Cesarean-delivered fetal membranes. The prepared, sealed, labeled, recorded, and packaged donor fetal membranes are then delivered to the separate tissue processing facility, as described above.

Immediately upon receipt at the processing facility, the shipping label is examined and information regarding the specimen and donor is recorded. The shipping container is examined for integrity, including confirmation of an intact tamper-proof seal. The shipping container is then opened and the inner bag containing the placental membranes and amniotic fluid is examined. An infrared temperature sensor is directed at the tissue bag to confirm a temperature of between 6 and 10 degrees Celsius. If there is any indication of damage to the outer container, the inner bag containing the placental membranes and amniotic fluid is examined with particular care. If damage to the inner bag is identified or the tamper-proof seal is broken or damaged, the specimen is not used to prepare the tissue graft. A donor/specimen data sheet within the container is then reviewed to validate the donor's credentials. The information on the data sheet is compared to the donor ID on the specimen bag to confirm the data sheet for the donor matches the specimen. This information is recorded and included in the permanent batch record for that specific donor. These credentials include donor lot numbers and expiration dates. All validation dates and times are confirmed. A donor tissue specimen that is unacceptable for any reason is discarded. The date, time, and hospital from which the donor specimen was received is recorded. The outside of the bag containing the two separate sterile specimen containers is then sprayed with isopropyl alcohol and manually wiped down. The logged and cleaned specimen bag containing the donor placental membranes and amniotic fluid is then stored in a locked refrigerator in an ice water bath, but not frozen.

Following first step 210 and receipt of the donor tissue, second step 220, comprises cleaning and preparation of the amniotic membrane for grinding or morcellizing, as practiced in some embodiments shown in FIG. 2. Under strict sterile technique, the specimen bag is opened using sterile scissors and the donor specimen comprising placental membranes and amniotic fluid is carefully poured into a large sterile basin. Using sterile forceps, the AM is peeled from the CM, which separates at the AM basement membrane/CM stromal interface. The AM is placed on a sterile cutting board, CM-side facing up. The CM side is gently wiped with sterile cloth towels, taking care to remove any adherent bits of CM and clotted blood which may not have been completely rinsed from the AM immediately following the delivery prior to packaging. Both sides of the AM are once again washed with sterile 0.9% saline and rinsed with an antimicrobial solution in some embodiments, such as 0.5% aqueous solution of glutaraldehyde for example.

After cleaning and preparation of the donor amnion, the next processing steps are generally divided into three groups which may be performed sequentially and in any order. Is sufficient number of personnel are available, all three groups of tissue processing steps may be executed simultaneously. One group of processing steps (Group I) involves treatment of the donor amnion to separate viable amniocytes from the stromal elements. A second group (Group II) involves creation of a partially dried amnion stromal particulate by morcellizing, grinding, shredding, or milling. A third group (Group 3) involves creation of an amniotic fluid derivative. These general groups of processing steps are shown in FIG. 3.

Group I, Separation of Amniocytes from Stromal Elements shown in FIG. 3, in some embodiments, comprises multiple procedures under step 225, preparation of sonicated amnion suspension shown in FIG. 2. The moist amnion from step 220 above is used. The amnion, in some embodiments, is divided using sterile instruments, scissors and forceps for example, measured into approximately equal volumes and placed 50 ml centrifuge tubes. In some embodiments, approximately half the amnion from a single donor is sonicated and half is morcellized, ground, or otherwise mechanically processed without sonication. In some embodiments, up to all the amnion is sonicated. In some embodiments, less than half the amnion is sonicated and the remainder is mechanically processed without sonication.

In some embodiments, a volume of trypsin solution is added. For example, an equal volume of 0.35% trypsin and amnion is placed in each tube. Other concentrations of trypsin, ranging from 0.025% or less to 0.50% or greater may be used in some embodiments. The trypsin solution is created by diluting a standardized stock solution with a buffered balanced electrolyte solution, such as a phosphate-buffered saline (“PBS”) solution for example. In some embodiments, no trypsin is used and the amnion is diluted with an equal volume of a sterile, non-pyrogenic isotonic solution of sodium chloride, sodium gluconate, sodium acetate, potassium chloride, and magnesium chloride buffered to a pH of 7.4 with sodium hydroxide (i.e., Plasma-Lyte A™, manufactured by Baxter International, Inc., Deerfield, Ill.).

The centrifuge tubes are sealed and sonicated. In some embodiments wherein a trypsin solution is used, the sonication is performed at a controlled temperature of 37°. In some embodiments, the sonication is performed using a commercially available sonicator (Crest Ultrasonics Digital Sonicator, product code CP360D, for example) for 1-3 hours at 45 kHz. Following sonication, the centrifuge tubes containing sonicated amnion are placed in a centrifuge. In some embodiments, the tubes are then centrifuged at 400-600 RPM for 10 minutes. In some embodiments, the tubes are centrifuged at a controlled temperature of between 6° and 10° Celsius. The tubes are then removed from the centrifuge and the supernatant decanted off the pellet and discarded. In some embodiments, the pellet containing viable SCs, protein, polypeptides, and other biologically active molecules is washed one or more times by re-suspending the pellet in a quantity of Plasma-Lyte, for example, pipetting the suspension into a new, sterile centrifuge tube, sealing the tube, and centrifuging at 400-600 RPM for 10 minutes. The tube is removed from the centrifuge, the supernatant is decanted off and discarded and the pellet is re-suspended in Plasma-Lyte, for example. This cycle may be repeated one or more additional times, in some embodiments. After decanting the supernatant off the pellet following the final cycle, in some embodiments, the pellet is re-suspended in 20 ml of Plasma-Lyte, for example, and is now referred to as Sonicated Amnion Solution (“SAS”).

A small aliquot, around 0.20 ml, is drawn from the SAS suspension and used for a cell count. Cell counts are performed, in some embodiments, to determine the concentration of viable SCs per volume of SAS. This enables preparation of a denuded amniotic membrane flowable tissue graft with a known, standardized SC component for use in applications described herein. Trypan Blue dye is used to determine cell viability. In some embodiments, a 0.5 ml 0.4% Trypan Blue solution is added to the 0.2 ml SAS suspension and mixed thoroughly by tapping the side of the tube. Hanks' Balanced Salt Solution, 0.3 ml, is added and the tube is further agitated to mix the contents. The tube is allowed to stand for 5-10 minutes, but not greater than 15 minutes to prevent uptake of dye by viable cells which may occur during longer exposure times. After staining, a small amount of stained suspension is pipetted into the counting chamber of a hemocytometer slide, a coverslip is applied and a cell count of unstained, viable SCs is performed using standardized techniques to obtain a cell count which accurately represents the concentration of viable SCs in the sampled SAS preparation.

Group II, Creation of Amnion Particulate shown in FIG. 3, in some embodiments, comprises steps 240 and 250 of overview 200 shown in FIG. 2. As shown in FIG. 2, step 240 comprises drying of the AM as practiced in some embodiments of the invention. Step 240 is employed in some embodiments prior to step 250 described herein below. In some embodiments a fresh, un-dried or partially dried AM is used in step 250. Using sterile scissors, the cleaned and treated AM from step 220 is place flat using sterile technique on a sterile lab board. A section of sterile mesh, in some embodiments, is gently spread over the AM and excess mesh is trimmed from around the edges of the AM. The AM/mesh is then placed on disinfected drying racks and allowed to dry for 10-20 minutes at ambient temperature. In some embodiments, the AM/mesh is placed in a drying oven at a controlled temperature for a controlled time, such as at 30° C. for 10 minutes for example. After the AM is completely dry to visible inspection, the AM/mesh is removed from the drying rack and the AM is gently peeled from the mesh for milling, morcellizing, or grinding described under step 250 herein below. In some embodiments, the mesh is a polyester mesh. In some embodiments, the mesh is a polypropylene mesh. These examples are not meant to be limiting; mesh formed from other synthetic materials suitable for implantation (i.e., hypoallergenic, non-pyrogenic) may also be used.

Step 250, in some embodiments, comprises milling, morcellizing, or grinding the AM. In some embodiments, the now dried AM is then removed from the drying rack and placed in a temperature-controlled ball-grinding mill (i.e. “CryoMill” for cryogenic grinding, manufactured by Retsch Corporation, Haan, Germany). The grinding jar and balls for the mill are weighed prior to placement of a quantity of dried AM in the grinding jar. After placement in the grinding jar, the dried AM is pre-cooled to minus 196° Celsius and then ground for approximately 4 minutes. This process results in an AM particle size of approximately 5 microns. In some embodiments, grinding proceeds for longer than 4 minutes, resulting in smaller particle size. In some embodiments, grinding proceeds for less than four minutes, resulting in larger particle size. The grinding jar is again weighed, and the weight of ground AM contained within is determined. In some embodiments, fresh AM us placed in the grinding jar for freezing and grinding without first drying the AM. In embodiments wherein this and other cryo-grinding procedures are employed, essentially all of the viable cellular elements of the AM, including SCs, are presumed lysed and no longer viable. Regardless, lysis of SCs and other cellular elements releases proteins, polypeptides, and other biologically active molecules into the AM particulate which are later suspended in the flowable tissue graft and available to the graft recipient tissue.

In some embodiments, the AM is morcellized but not ground. A morcellized amnion may also comprise amniocytes which are lysed, along with non-lysed, viable amniocytes. Preparations with disrupted amniocytes have a higher concentration of growth factors, other functional protein and peptide molecules, and other biologically active molecules which are released into the preparation from the disrupted cells. Non-disrupted viable amniocytes comprise SCs which become engrafted into host tissue and participate in tissue regeneration and may lend additional or other beneficial, therapeutic effects to the tissue graft.

In these and similar embodiments utilizing morcellized or ground AM, fresh, dried, or partially dried AM is cut into approximately 1 cm-wide strips using tissue scissors under sterile conditions. The cut strips of AM are then ground or morcellized using a variable speed tissue homogenizer at between 500 and 1000 rpm for a brief, limited time. Grinding or morcellizing is stopped when the AM is grossly shredded into tiny pieces by visual inspection, typically after no longer than five to fifteen seconds, depending upon the amount of specimen being processed. The individual pieces are visible and fall within in an approximate range of 0.1 mm to 1.0 mm in size. The ground or morcellized AM is then completely dried in some embodiments. In some embodiments, the ground or morcellized AM is dried in a sterile container within a drying oven at controlled temperature for a set time. In some embodiments, the ground or morcellized AM is dried in a sterile container under ambient conditions.

In some embodiments, AM which has been previously processed, such as in a dried, partially dried, or fresh state; packaged; and sterilized by irradiation is used for grinding or morcellizing.

Group III, Creation of Amniotic Fluid Derivative shown in FIG. 3, in some embodiments, comprises multiple steps under step 230 shown in FIG. 2. Step 230 comprises removing AF from the large sterile basin in 50 ml aliquots using a sterile pipette. Each 50 ml aliquot of AF is pipetted into a centrifuge tube, which is capped and then spun-down into a pellet and supernatant by centrifuging at 1,200 RPM for 10 minutes, in some embodiments. The supernatant, containing water, electrolytes, and other solutes, is pipetted from the tube and discarded. The pellet, containing SCs, proteins, polypeptides, and other large molecules with associated insoluble compounds is reconstituted in a suitable fluid. In some embodiments, the suitable fluid is a sterile buffered isotonic electrolyte solution, such as Plasma-Lyte. In some embodiments, the suitable fluid is a cryoprotectant. In some embodiments, the suitable fluid is a 10% solution of dimethylsulfoxide (“DMSO”). In some embodiments, the suitable fluid is a 5% solution of DMSO. In some embodiments, the suitable fluid is a 50% solution of glycerol. A quantity of this solution just adequate to suspend the pellet material for removal by pipette is used, usually one ml or less. The washed, re-constituted AF material from two tubes in the first spin-down is combined into one centrifuge tube and centrifuged for a second time at 1,200 rpm for 10 minutes. Again, the supernatant is pipetted off the solid pellet at the bottom of the tube, which is again re-constituted in a fresh quantity of suitable fluid, pipetted from the tube, and combined with a second washed re-constituted specimen. This sequence is repeated a third time, after which the centrifuge tube contained the thrice-washed pellet and supernatant is re-constituted in a quantity (1.0 ml to 5.0 ml, for example; not meant to be limiting) of fresh suitable fluid and the centrifuge tubes are temporarily stored in a water-ice bath.

Prior to storage, a 0.2 ml sample from each lot of washed, re-constituted processed AF derivative is removed for a cell count using Trypan Blue and a hemocytometer described herein above. The number of grossly viable SCs/cc of suspension is calculated and recorded for later standardization of SC concentration per unit volume of the final injectable amniotic tissue graft preparation.

In some embodiments, the sealed, sterile plastic specimen container with AF is refrigerated but not frozen, and is not centrifuged and washed as described above. In some embodiments, the AF is combined with a cryoprotectant, such as DMSO at a 5% concentration by weight, frozen at a controlled rate to −80° C., and stored for later thawing and use in forming a denuded amniotic membrane flowable tissue graft. In some embodiments, a 10% solution of DMSO is used. The use of DMSO is not meant to be limiting. Other suitable cryoprotectants, such as a solution of 50% glycerol for example, may be used. In some embodiments, cryogenic tubes containing the washed AF-cryoprotectant preparation are placed in a controlled rate freezer. A single tube is selected for placement of a temperature monitoring probe; the contents of this tube will be discarded after freezing. In some embodiments, the AF preparation is frozen at a rate of −2° per minute down to −80° C. In some embodiments, the preparation is frozen at a rate of −4° per minute down to −80° C.

Step 260, in some embodiments, comprises mixing the milled, ground, or morcellized AM with AF, centrifuged and decanted AF, or processed AF derivative; and a quantity of SAS to form the denuded amniotic membrane tissue graft. In some embodiments, ground AM is hydrated with AF, centrifuged and decanted AF, or processed AF derivative, with or without a second suitable fluid, by mixing step 260.

In some embodiments, the weighed grinding jar containing the dried, ground AM is opened an the ground AM is washed from the jar and balls using a quantity of suitable fluid, approximately 50 ml of sterile isotonic saline, for example. In some embodiments, the ground AM/fluid (“AMFL”) is placed in a small sterile beaker and an autoclaved sterile Teflon-coated mixing magnet is placed in the AMFL. The beaker is then placed on a mixer and mixed for a few minutes using a low RPM setting in order to remove any small or microscopic bits of metal which may have been introduced to the AM during the grinding process.

In some embodiments, the weight of AM per volume of denuded amnion flowable tissue graft is standardized. Prior to mixing step 260, the weighed grinding jar containing the dried, ground AM is opened and the ground AM is washed from the jar and balls using a quantity of suitable fluid, approximately 50 ml of sterile isotonic saline solution, for example. Just enough solution is added to liquefy and partially reconstitute the ground AM. The exact quantity is recorded so that in addition to a standard cell count (based upon the initial donor cell count of SCs/ml of the re-constituted processed AF derivative or other hydrating fluid described in step 230 above), a standardized concentration by weight of AM per unit volume of re-constituted AF is also provided for the completed flowable tissue graft, in some embodiments of the invention. The reconstituted AM in reconstituted AF (or other suitable fluid) (“AMFL”) therefore, has a known weight of AM per volume of AMFL. In some embodiments, the concentration by weight of AM per unit volume of tissue graft is chosen to form a tissue graft of a desired viscosity. In some embodiments, a suitable gelling agent, such as a prepared collagen gel for example, is added to the denuded amnion tissue graft to create a high-viscosity fluid or gel consistency.

In some embodiments, step 260 comprises reconstitution of the ground AM with fresh, unmodified AF. In some embodiments, centrifuged, unwashed AF from which a portion of the supernatant has been decanted is used, such that the volume of centrifuged, decanted AF is the volume necessary to create a desired standardized concentration by weight of ground AM in the formed denuded amnion tissue graft product. In some embodiments, cryopreserved AF is used. In some embodiments, the AF or AF preparation is from the same donor as the AM. In some embodiments, the AF or AF preparation is from a different donor as the AM. In some embodiments, the AF, centrifuged decanted AF, or processed AF derivative is from pooled multiple donors. In some embodiments, the ground AM is from pooled multiple donors. In some embodiments, the AF is from a non-human mammalian species. In some embodiments, the AM is from a non-human mammalian species.

Step 260 is completed by combining quantities of AMFL, washed and re-constituted AM, a cryoprotectant, and buffered isotonic solution to form the completed tissue graft. Sterile materials are used and sterile technique is maintained. In some embodiments, a previously recorded weight per volume of AM and SC per volume of AF are noted such that the completed tissue graft is a known, standardized, reproducible product. In some embodiments, a previously recorded number of viable SCs per unit volume, as described in step 230 of FIG. 2, are calculated by counting grossly viable SCs in an aliquot of AMFL/AF using Trypan Blue and a hemocytometer as described herein above or other suitable cell counting method, and using dilution tables to calculate the final concentration of SCs per unit volume of standardized denuded amnion flowable tissue graft 100.

An example buffered isotonic solution used in some embodiments to create the AMFL and reconstituted AF is Plasma-Lyte A An example of a cryoprotectant used in some embodiments is CryoStor CS-10, a 10% solution of DMSO (manufactured by BioLife Solutions, Inc., Bothel, Wash.). These examples are not meant to be limiting; similar products may be compounded or obtained from other manufacturers for use in preparation of the tissue graft. Standardized dilution tables are pre-calculated based upon the initial donor cell count completed previously in step 230. Once the final dilution ratios have been confirmed and prepared, the measured individual components are poured into a large beaker and gently suspended by gently swirling the beaker and/or stirring with a glass rod or other suitable instrument.

In some embodiments, a small quantity of denuded amnion flowable tissue graft 100, approximately 0.2 cc's for example, is drawn into a sterile 2 cc syringe and extruded through a 25 gauge or smaller needle to ensure the tissue graft is sufficiently fluid to be percutaneously or intraoperatively injected/deposited into the recipient tissue bed. In some embodiments, the viscosity of the tissue graft is further adjusted by mixing an additional measured quantity of buffered isotonic solution, such as or similar to Plasma-Lyte, with the tissue graft and recording the final concentration of AM and SC per ml accordingly. In some embodiments, the final concentration of AM and/or SC per ml is adjusted with additional buffered isotonic solution to an end-user's pre-ordered concentration requirement based upon the intended use of standardized denuded amnion flowable tissue graft 100.

In some embodiments, step 260 also comprises determining the quantity of finished tissue graft requested by end user based upon the intended use of the completed denuded amnion flowable tissue graft. In some embodiments, the denuded amnion flowable tissue graft is packaged in standard SC concentrations, AM concentrations, and total volumes. In some embodiments, the denuded amnion flowable tissue graft is packaged in standard differing viscosities based upon the mode used for delivery (injection versus intraoperative application, for example) and intended therapeutic use.

In some embodiments, the denuded amnion flowable tissue graft 100 is pipetted into empty product vials and placed in a lyophilization unit for controlled removal of water and other volatiles prior to final packaging and storage. The packaging vials of lyophilized tissue graft are then sterilely sealed, labeled, and cooled in a controlled-rate freezer to minus 80° Celsius, as in the example procedures discussed herein above in some embodiments.

In some embodiments, the denuded amnion flowable tissue graft 100 containing a cryoprotectant, such as the examples listed herein, is frozen in a controlled-rate freezer without lyophilization prior to freezing.

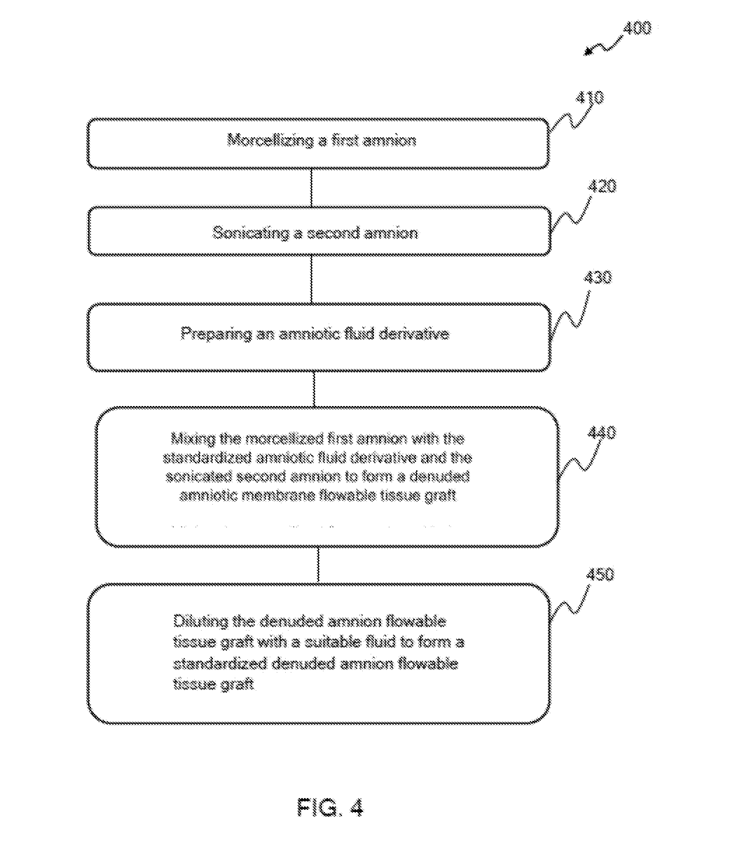

FIG. 4 shows a method 400 of forming some embodiments of denuded amnion flowable tissue graft 100. Method 400 comprises morcellizing step 410, sonicating step 420, preparing step 430, and mixing step 440. In some embodiments, including the embodiment shown in FIG. 4, method 400 further comprises optional diluting step 450 comprising diluting the denuded amnion flowable tissue graft with a suitable fluid to form a standardized denuded amnion flowable tissue graft.

Morcellizing step 410 of method 400 is performed on a fresh, dried, or partially dried amnion, and some embodiments have been described herein (see description of overview 200, steps 240 and 250). Sonicating step 420 of method 400 is performed on a fresh, fully hydrated amnion, and some embodiments have been described herein (see description of overview 200, steps 220 and 225). In some embodiments, method 400 further comprises a digesting step (not shown) wherein an enzyme is used to facilitate separation of the amniocyte SCs from the amnion stromal elements, and some embodiments have been described herein (see overview 200, preparing step 225). In some embodiments, the enzyme used is trypsin. Preparing step 430 comprises processing fresh amniotic fluid to form an amniotic fluid derivative, and some embodiments have been described herein. Some embodiments of processing steps comprising preparing step 430 have been described herein (see overview 200, preparing step 230). Mixing step 440 comprises mixing the three preparations: morcellized first amnion, sonicated amnion suspension, and amniotic fluid derivative together to form denuded amniotic membrane flowable tissue graft 100 and some embodiments have been described herein (see overview 200, mixing step 260).

Step 410 of method 400 comprises morcellizing or grinding an amnion, e.g. using a cryomill as described herein. In some embodiments, fresh, non-dried AM is frozen in a cryomill immediately prior to morcellizing. In some embodiments, a dried or partially dried AM is frozen in the cryomill immediately prior to morcellizing. In some embodiments, step 410 is performed by morcellizing an amnion by a means described herein above or by another means known in the art. Specific details of various means used to perform step 410 of method 400, in some embodiments of the invention have been disclosed and described herein above.

Step 430 of method 400 shown in FIG. 4 comprises preparing an amniotic fluid derivative, in some embodiments. In some embodiments, amniotic fluid derivative is fresh amniotic fluid without further processing or addition of other material. In some embodiments, amniotic fluid derivative is processed according to one of the means described herein above. In some embodiments, amniotic fluid derivative is a centrifuged and decanted quantity of amniotic fluid. In some embodiments, amniotic fluid derivative is an amniotic fluid preparation to which a biologically active material, such as hyaluronic acid for example, has been added. In some embodiments, amniotic fluid derivative is processed according to a suitable means known in the art but not described herein.

Step 430 of method 400 shown in FIG. 4 comprises quantifying a concentration of viable mesenchymal stem cells in the amniotic fluid derivative to form a standardized amniotic fluid derivative. As discussed herein above (see FIG. 2, step 230), in some embodiments, a 0.5 ml sample from each lot of washed, re-constituted processed AF derivative is removed for a cell count, using techniques known in the art, prior to storage of the AF derivative. The number of grossly viable SCs/cc of suspension is calculated and recorded for standardization of SC concentration per unit volume of the final injectable amniotic tissue graft preparation.

FIG. 4 also shows step 440 of method 400 comprising mixing the ground amnion with a quantity of processed amniotic fluid derivative to form standardized injectable amniotic tissue graft. In some embodiments, processed amniotic fluid derivative is fresh amniotic fluid collected under sterile conditions and refrigerated without freezing, without further processing or addition of other material. In some embodiments, processed amniotic fluid derivative is a processed, concentrated amniotic fluid reconstituted with a suitable fluid following serial washings as discussed herein above. The processed amniotic fluid derivative mixed with ground amnion in step 440 determines the final desired concentration of viable SCs/ml of standardized injectable denuded amnion flowable tissue graft 100 desired in the completed product. This is calculated based upon the previously recorded number of grossly viable SCs suspended/cc in the lot of AF derivative used.

Method 400 further comprises optional diluting step 450; namely, diluting the standardized denuded amnion flowable tissue graft with a suitable fluid to form a second standardized denuded amnion flowable tissue graft. Various suitable fluids have been defined herein above, including but not limited to buffered isotonic electrolyte solution(s), cryoprotectant solutions, and other solutions. In some embodiments, no additional suitable fluid is added. In some embodiments, additional suitable fluid is added in an amount to achieve the desired end concentration of viable SCs/ml of standardized denuded amnion flowable tissue graft 100. In some embodiments, standard dilution tables are used to determine the amount of suitable fluid added.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above, and are intended to fall within the scope of the appended claims.

What is claimed is:

1. A denuded amnion flowable tissue graft comprising:
an amniotic membrane stromal matrix denuded of amniocytes; and
a non-amniotic fluid liquid, wherein the non-amniotic fluid liquid hydrates the amniotic membrane stromal matrix.

2. The tissue graft of claim 1, wherein the non-amniotic fluid liquid is an isotonic electrolyte solution.

3. The tissue graft of claim 1, wherein the non-amniotic fluid liquid is a cryoprotectant.

4. The tissue graft of claim 1, wherein the non-amniotic fluid liquid comprises an isotonic electrolyte solution and a cryoprotectant.

5. The tissue graft of claim 1, further comprising an amniotic fluid derivative.

6. The tissue graft of claim 1, wherein the denuded amniotic membrane is sonicated.

7. The tissue graft of claim 1, wherein the denuded amniotic membrane is morcellized.

8. The tissue graft of claim 1, wherein the denuded amniotic membrane is ground.

9. A denuded amnion flowable tissue graft comprising:
an amniotic membrane stromal matrix denuded of amniocytes;
a non-denuded amniotic membrane comprising an amniocyte layer; and
a non-amniotic fluid liquid, wherein the non-amniotic fluid liquid hydrates the amniotic membrane stromal matrix.

10. The tissue graft of claim 9, wherein the non-denuded amniotic membrane is morcellized.

11. The tissue graft of claim 9, wherein the non-denuded amniotic membrane is ground.

12. The tissue graft of claim 9, wherein the non-denuded amniotic membrane is sonicated.

13. A denuded amnion flowable tissue graft comprising:
an amniotic membrane stromal matrix denuded of amniocytes;
an amniotic fluid derivative, wherein the amniotic fluid derivative hydrates the amniotic membrane stromal matrix; and
a sonicated amnion suspension.

14. The tissue graft of claim 13, further comprising a known concentration of viable mesenchymal stem cells.

15. The tissue graft of claim 14, wherein the concentration of viable mesenchymal stem cells is less than 5.0×10⁵/ml.

16. The tissue graft of claim 14, wherein the concentration of viable mesenchymal stem cells is between 5.0×10⁵/ml and 1.0×10⁶/ml.

17. The tissue graft of claim 14, wherein the concentration of viable mesenchymal stem cells is between 1.0×10⁶/ml and 1.50×10⁶/ml.

18. The tissue graft of claim 14, wherein the concentration of viable mesenchymal stem cells is greater than 1.5×10⁶/ml.

19. A set of tissue grafts wherein each tissue graft in the set comprises:
an amniotic membrane stromal matrix denuded of amniocytes;
an amniotic fluid derivative wherein the amniotic fluid derivative hydrates the amniotic membrane stromal matrix;
a sonicated amnion suspension; and
a known concentration of viable mesenchymal stem cells

20. The set of tissue grafts of claim 19, wherein the concentration of viable mesenchymal stem cells is less than 5.0×10⁵/ml.

21. The set of tissue grafts of claim 19, wherein the concentration of viable mesenchymal stem cells is between 5.0×10⁵/ml and 1.0×10⁶/ml.

22. The set of tissue grafts of claim 19, wherein the concentration of viable mesenchymal stem cells is between 1.0×10⁶/ml and 1.5×10⁶/ml.

23. The set of tissue grafts of claim 19, wherein the concentration of viable mesenchymal stem cells is greater than 1.5×10⁶/ml.

24. The set of tissue grafts of claim 19 comprising at least 10 tissue grafts.

25. The set of tissue grafts of claim 19 comprising at least 50 tissue grafts.

26. A method of forming a denuded amnion flowable tissue graft comprising the steps of:
morcellizing a first amnion;
sonicating a second amnion;
preparing an amniotic fluid derivative; and
mixing the morcellized first amnion with the sonicated second amnion and the amniotic fluid derivative to form a denuded amnion flowable tissue graft.

27. The method of claim 26, further comprising a step diluting the denuded amnion flowable tissue graft with a suitable fluid to form a standardized denuded amnion flowable tissue graft.
